(19) United States
(12) Patent Application Publication
(10) Pub. No.: US 2022/0068480 A1
(43) Pub. Date: Mar. 3, 2022

(54) SYSTEMS AND METHODS FOR PREDICTING AND IMPROVING THE HEALTHCARE DECISIONS OF A PATIENT VIA PREDICTIVE MODELING

(71) Applicant: FOUNDRYDC, LLC, Washington, DC (US)

(72) Inventor: James MANZI, Washington, DC (US)

(21) Appl. No.: 17/411,102

(22) Filed: Aug. 25, 2021

Related U.S. Application Data
(60) Provisional application No. 63/072,590, filed on Aug. 31, 2020.

(51) Int. Cl. G16H 50/20; G16H 50/30; G16H 40/20; G16H 10/60; G16H 50/70; G16H 30/20; G16H 30/40; G06T 7/00

(52) U.S. Cl. CPC G16H 50/20; G16H 50/30; G16H 40/20; G16H 10/60; G16H 50/80; G16H 30/20; G16H 30/40; G06T 7/0012; G16H 50/70

(57) ABSTRACT

A system and method for predicting and improving the healthcare decisions of a patient via predictive modeling. The system and method include receiving a request for a risk score; applying scoring data to a risk predictive model causing the risk predictive model to generate the risk score, the risk score indicative of a probability of the patient to access in-person medical care at a medical provider within a temporal window; determining that the risk score satisfies a criteria; and applying at least a subset of the scoring data to an impactability predictive model causing the impactability predictive model to generate an impactability score based on at least the subset of the scoring data, the impactability score indicative of a probability that the patient would not access the in-person medical care at the medical provider within the temporal window responsive to receiving a notification from the medical provider.

### CROSS REFERENCE TO RELATED APPLICATIONS

This application claim priority to U.S. Provisional Application No. 63/072,590, filed Aug. 31, 2020, which is incorporated by reference herein in its entirety for all purposes.

### TECHNICAL FIELD

This application relates generally to artificial intelligence in the field of computer science, and more particularly to systems and methods for predicting and improving the healthcare decisions of a patient via predictive modeling.

### BACKGROUND

Predictive analytics is a data mining technique that attempts to predict an outcome. Predictive analytics uses predictors or known features to create predictive models that are used in obtaining an output. A predictive model reflects how different points of data interact with each other to produce an outcome. Predictive modeling is the process of using known results to create, process, and validate a model that can be used to forecast future outcomes.

Healthcare is the maintenance or improvement of health via the prevention, diagnosis, treatment, recovery, or cure of disease, illness, injury, and other physical and mental impairments in people. The healthcare industry includes the medical providers (e.g., doctors, hospitals) who receive a fee for providing medical care to patients. The medical providers often have the most interaction with the patients and are best equipped to influence the behavior of the patients. For example, a doctor may be able to encourage a patient to come in for a medical check-up, attend a counseling program to reduce weight, or to have surgery.

The healthcare industry also includes the health insurance companies who bear all the risk and expense for the provided medical care. Unlike the medical providers, the health insurance companies have limited interaction with the patients and much less capability to influence the patient behavior.

For these reasons, the medical providers and health insurance companies have spent considerable resources producing software solutions having various models to predict the risks associated with insuring patients. For instance, conventional software solutions utilized by the medical providers may use algorithmic approaches to calculate a likelihood of a patient having a follow up visit subsequent to a medical procedure. While the results produced by conventional software solutions are helpful, they are also incomplete. Conventional software solutions rely on patient attributes to produce results. However, lack of data associated with other patients has caused conventional software solutions to produce ineffective and sometimes inaccurate results. Moreover, simply adding data associated with other patients to be considered by conventional software solutions is not viable solutions because of the following three reasons.

First, the data may not be readily identifiable/ingestible by conventional software solutions. For instance, some healthcare data needs to be pre-processed and analyzed before being ingested by an algorithm. Second, healthcare data may be stored onto different platforms and managing such information on different platforms is difficult due to number, size, content, or relationships of the data associated with the customers. Third, conventional software solutions use static algorithms and produce the same result for all patients. Therefore, these software solutions are unable to produce accurate results that account for peculiar and individual patient attributes. Instead, conventional software solutions follow the same path for all patients.

### SUMMARY

For the aforementioned reasons, there is a long-felt desire in designing solutions that model both risk and impactability in order to optimize the deployment of diagnostic and engagement services. Disclosed herein are methods and system design to analyze healthcare-specific data (for a particular patient and other patients) and execute multiple artificial intelligence models to achieve meaningful results.

In an embodiment, a method comprises receiving, by one or more processors, a request for a risk score associated with a patient of one or more medical providers; applying, by the one or more processors, scoring data associated with the patient to a risk predictive model that is trained with training data causing the risk predictive model to generate the risk score based on the scoring data, the risk score indicative of a probability of the patient to access in-person medical care at a medical provider within a temporal window that is subsequent to the one or more processors receiving the request for the risk score; responsive to determining that the risk score satisfies a criteria, applying, by the one or more processors, at least a subset of the scoring data to an impactability predictive model to generate an impactability score indicative of a probability that the patient would not access the in-person medical care at the medical provider within the temporal window responsive to receiving a notification from the medical provider; and sending, by the one or more processors, a message to a client device instructing the client device to present at least one of the risk score or the impactability score.

In another embodiment, a system comprises a server comprising a processor and a non-transitory computer-readable medium containing instructions that when executed by the processor causes the processor to perform operations comprising: receive a request for a risk score associated with a patient of one or more medical providers; apply scoring data associated with the patient to a risk predictive model that is trained with training data causing the risk predictive model to generate the risk score based on scoring data, the risk score indicative of a probability of the patient to access in-person medical care at a medical provider within a temporal window that is subsequent to the processor receiving the request for the risk score; responsive to determining that the risk score satisfies a criteria, apply at least a subset of the scoring data to an impactability predictive model to generate an impactability score indicative of a probability that the patient would not access the in-person medical care at the medical provider within the temporal window responsive to receiving a notification from the medical provider; and send a message to a client device instructing the client device to present at least one of the risk score or the impactability score.

These and other features, together with the organization and manner of operation thereof, will become apparent from the following detailed description when taken in conjunction with the accompanying drawings.

### BRIEF DESCRIPTION OF THE FIGURES

FIG. 1 is a block diagram depicting an example environment for predicting and improving the healthcare decisions of a patient via predictive modeling, according to some embodiments.

FIG. 2A is a block diagram depicting an example analytical management system, according to some embodiments.

FIG. 2B is a block diagram depicting an example predictive model server, according to some embodiments.

FIG. 2C is a block diagram depicting an example notification system, according to some embodiments.

FIG. 3 is a flow diagram depicting a method for predicting and improving the healthcare decisions of a patient via predictive modeling, according to some embodiments.

FIG. 4 is a graphical user interface of an example application depicting a method for displaying a plurality of risk models, according to some embodiments.

FIG. 5 is a graphical user interface of an example application depicting a method for selecting an outcome for a risk model, according to some embodiments.

FIG. 6 is a graphical user interface of an example application depicting a method for searching procedure codes and code ranges for a risk model, according to some embodiments.

FIG. 7 is a graphical user interface of an example application depicting a method for selecting a predetermined temporal window as a condition for executing a risk model, according to some embodiments.

FIG. 8 is a graphical user interface of an example application depicting a method for filtering training data and/or scoring data for executing a risk model, according to some embodiments.

FIG. 9 is a graphical user interface of an example application depicting a method for displaying a plurality of care access features for a risk model, according to some embodiments.

FIG. 10 is a graphical user interface of an example application depicting a method for displaying the output predictions and/or model results from a risk model, according to some embodiments.

FIG. 11 is a graphical user interface of an example application depicting a method for displaying a plurality of impactability models, according to some embodiments.

FIG. 12 is a graphical user interface of an example application depicting a method for displaying the output predictions and/or model results from an impactability model, according to some embodiments.

### DETAILED DESCRIPTION

The systems and methods for predicting and improving the healthcare decisions of a patient via predictive modeling. In one aspect, a risk predictive model can determine the probability of a patient to access in-person medical care at a medical provider during a predetermined time frame. In another aspect, an impactability predictive model can determine the probability of the medical provider to impact whether the patient accesses the in-person medical care at the medical provider during the predetermined time frame. The embodiments disclosed herein solve the aforementioned problems and other problems.

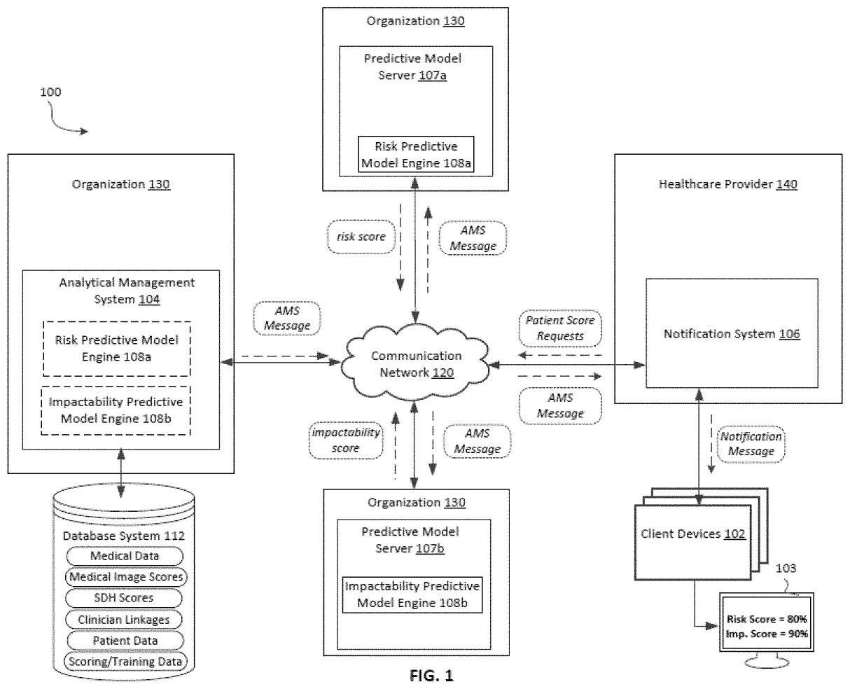

In general, as described in the below passages and specifically in the description of FIG. 1, an organization (e.g., a healthcare provider, a data modeling service provider, a cloud service provider) may operate an analytical management system that performs a series of operations (e.g., processes, tasks, actions) associated with a risk predictive model engine and an impactability predictive model engine that each execute on the analytical management system and/or on one or more predictive model servers. These operations may be categorized into two phases: a “Training Phase” for training the predictive model engines and a “Management Phase” for managing the performance of the predictive model engines, once trained.

During the Training Phase, the analytical management system trains (e.g., creates, builds) the risk predictive model engine to generate a risk score indicating the probability that a patient (e.g., as identified by a patient identifier) of a medical provider will access in-person medical care at the medical provider during a predetermined time frame (e.g., within the next 6 months). Conversely, the analytical management system trains the impactability predictive model engine to generate an impactability score indicating the probability of the medical provider to impact (e.g., influence, control, steer) whether the patient accesses the in-person medical care at the medical provider during the predetermined time frame subsequent to receiving a notification (e.g., a phone call, an email, a postal letter) from the medical provider. While the output of the risk predictive model engine may be provided as an input to the impactability predictive model engine, each predictive model engine may, in some instances, operate separately and/or independently. For example, the impactability predictive model engine may determine (e.g., predict, forecast, estimate) whether a notification would deter a patient from accessing in-person medical care, where the risk predictive model engine had previously determined that this particular patient had a high likelihood of accessing the in-person medical care. Alternatively, the impactability predictive engine may determine whether a notification would deter a patient from accessing in-person medical care, where the risk predictive model engine had not yet been used to determine this particular patient's risk score.

The analytical management system generates (e.g., creates, builds, constructs) a first set of training data that it uses to train the risk predictive model engine and a second set of training data that it uses to train the impactability predictive model engine, where each set of training data consists of a plurality of input features (sometimes referred to as “input variables”). The analytical management system generates the first set of training data using medical data (sometimes referred to as “clinician data,” e.g., electronic health records, insurance claim charts, lab records, prescriptions) that was acquired (e.g., gathered, collected) by one or more healthcare providers (e.g., medical service providers, health insurance providers) from one or more patients, medical image scores, social determinants of health (SDH) scores, and/or clinician linkages (sometimes referred to as “clinician graph data”).

The analytical management system generates the second set of training data based on a plurality of impactability identifiers that are associated with a plurality of patients. Each impactability identifier indicates that a healthcare provider (e.g., a physician's office, an insurance company) was able to successfully impact a patient's decision to not access in-person medical care by sending a notification to the patient. For example, a healthcare provider may discover (e.g., determine, learn) that a particular patient of a medical provider is planning to access in-person medical care from the medical provider within the next 2 weeks to inquire as to whether the patient's leg cast should be replaced with a new leg cast. The healthcare provider may discover this information, for example, based on receiving a risk score from a risk predictive model engine. Seeking to reduce the associated costs for providing the in-person medical care, the healthcare provider may decide to contact the patient by phone (or via email, postal service, text message) before the patient accesses the in-person medical care at the medical provider in order to provide the medical care over the phone and/or to impact the patient's decision to not access the in-person medical care. In some embodiments, the second set of training data may include one or more subsets (or all subsets) of the first set of training data.

The analytical management system may deploy (“bring on-line”) the now-trained, predictive model engines (e.g., risk predictive model engines and/or the impactability predictive model engine) into a production environment, such that the predictive model engines may each be relied on (together or separately/independently) by one or more computing systems (e.g., a notification system) of a healthcare provider for the purpose of generating predictions about the behavior of one or more patients of the healthcare provider. The analytical management system may deploy the predictive model engines into the production environment by executing one or more of the predictive model engines on the analytical management system, and/or by sending one or more messages to one or more predictive model servers to cause the one or more predictive model servers to execute the risk predictive model engines and/or the impactability predictive model engine. The predictive model servers may be operated/managed by the organization that operates the analytical management system or another organization (e.g., a data modeling service provider, a cloud service provider).

During the Management Phase, the analytical management system may receive a request (sometimes referred to as “a patient score request”) from an application (e.g., a web browser application, a custom software application, a software development kit (SDK)) executing on the notification system to generate a risk score indicating the probability of a patient of a medical provider to access in-person medical care at the medical provider within a predetermined temporal window (e.g., two weeks from receiving the request, six months from receiving the request). The request may include a patient identifier and/or a description of the predetermined temporal window. In response to receiving the request, the analytical management system may generate a first set of scoring data that is associated with the patient (e.g., the patient that was identified in the request) based on data that the analytical management system retrieves from a database. The first set of scoring data may include medical data associated with the patient, medical image scores associated with the patient, SDH scores associated with the patient, and/or clinician linkages associated with the patient.

The analytical management system may determine whether a risk predictive model engine is available to process the request—for example, whether a risk predictive model engine has already been trained and deployed into production (e.g., executing on the analytical management system or on a predictive model server). If the analytical management system determines that a risk predictive model engine is not available to process the request, then the analytical management system may train and deploy a risk predictive model engine into the production environment according to the operations of the Training Phase, as discussed herein.

Upon identifying a risk predictive model engine that is available to process the request, the analytical management system may apply (e.g., insert, provide) the first set of scoring data associated with the patient to the risk predictive model engine by sending a message (sometimes referred to as “an AMS message”) to the risk predictive model engine. The message, which includes the first set of scoring data, causes the risk predictive model engine to generate a risk score based on the first set of scoring data and send the risk score to the analytical management system. In response to receiving the risk score, the analytical management system may send a message to the notification system, where the messages causes the notification system to present the risk score on a display (e.g., computer screen, touchscreen) associate with notification system. In some embodiments, the message causes the notification system to send a second message to a client device to instruct the client device to present the risk score on a display associated with the client device.

In response to receiving the risk score, the analytical management system may determine whether the risk score satisfies a criteria (e.g., equal to and/or above or below a criteria) by comparing the risk score against the criteria. If the analytical management system determines that the patient has a high probability of accessing in-person medical care at the medical provider within a predetermined temporal window (e.g., 2 weeks from receiving the request, 6 months from receiving the request), then the analytical management system may proceed to determine whether an impactability predictive model engine is available to process a request. If an impactability predictive model engine is not available, then the analytical management system may deploy an impactability predictive model engine into the production environment according to the operations of the Training Phase, as discussed herein.

The analytical management system may generate a second set of scoring data that includes one or more subsets (or all subsets) of the first set of scoring data associated with the patient. The analytical management system may apply (e.g., insert, provide) the second set of scoring data to the impactability predictive model engine by sending a message (sometimes referred to as “an AMS message”) to the impactability predictive model engine. The message, which includes the second set of scoring data, causes the impactability predictive model engine to generate an impactability score based on the second set of scoring data and send the impactability score to the analytical management system.

In response to receiving the impactability score, the analytical management system may send a message (sometimes referred to as an, “AMS message”) to the notification system, where the messages causes the notification system to present the risk score and/or the impactability score on a display of the notification system. In some embodiments, the message causes the notification system to send a second message (sometimes referred to as a “notification message”) to a client device to cause the client device to present the risk score and/or the impactability score on a display of the client device.

Thus, an analytical management system may train and manage a risk predictive model engine and/or an impactability predictive model engine that may be relied on by a healthcare provider for predicting and/or improving the healthcare decisions made by the patients of the healthcare provider.

FIG. 1A is a block diagram depicting an example environment for predicting and improving the healthcare decisions of a patient via predictive modeling, according to some embodiments. The environment 100 includes an analytical management system 104 that is operated/managed by an organization 130 (e.g., a healthcare provider, a data modeling service provider, a cloud service provider) and configured to train and/or manage one or more predictive model engines.

The environment 100 includes a database system 112 that is communicably coupled to the analytical management system 104 for storing medical data, medical image scores, SDH scores, clinician linkages, any other patient data, scoring data, and training data. The analytical management system 104 may populate the database system 112 using information that is received (e.g., acquired, gathered, collected) by the organization 130 and/or any other organization (e.g., healthcare provider 140).

The medical data may include electronic health records, insurance claim charts, lab records (e.g., blood tests, cholesterol tests), and/or prescriptions (e.g., prescription name, prescription dosage, prescribing doctor). The electronic health record is the systematized collection of patient and population electronically-stored health information in a digital format.

Each medical image score is associated with a respective patient and indicates a probability that the patient has a medical illness. The medical scores may be generated by a predictive model engine (not shown in FIG. 1) based on a plurality of medical images (e.g., X-ray, computed tomography (CT) scan, magnetic resonance imaging (MRI)) and a plurality of medical diagnosis labels, where each medical image is associated with a respective medical diagnosis label. For example, a medical diagnosis label may indicate that an X-ray of a patient's lung shows that the patient has lung cancer.

The SDH factors are the economic and social conditions that influence individual and group differences in health status. They are the health promoting factors found in a person's living and working conditions (e.g., the distribution of income, wealth, influence, and power), rather than individual risk factors (e.g., behavioral risk factors or genetics) that influence the risk for a disease, or vulnerability to disease or injury. The SDH factors may be normalized down to the neighborhood level, such as down to the zip code and/or census track level. For example, an SDH factor may indicate the percentage of people in a neighborhood that get their colorectal scans on time. As another example, an SDF factor may indicate whether there are environmental threats in a neighborhood.

The clinician linkages each indicate a degree of relationship between a plurality of physicians. For example, a clinician linkage may indicate that Dr. Smith and Dr. Jones have a medical practice together at a particular address. As another example, a clinician linkage may indicate that Dr. Smith and Dr. Jones went to the same medical school.

The environment 100 includes predictive model servers 107a, 107b (collectively referred to herein as predictive model servers 107) that are in communication with the analytical management system 104 via a communication network 120. The predictive model servers 107a, 107b are operated/managed by the organization 130 and configured to execute (e.g., run, launch) a risk predictive model engine 108a and an impactability predictive model engine 108b, respectively. In some embodiments, the analytical management system 104 may execute the risk predictive model engine 108a and/or the impactability predictive model engine 108b.

The environment 100 includes a notification system 106 that is operated/managed by a healthcare provider 140 (e.g., insurance provider, medical provider). The notification system 106 is in communication with the analytical management system 104 and one or more client devices 102 via the communication network 120. The notification system 106 is configured to execute an application that allows a user of the notification system 106 to send (via the application) a risk score request and/or an impactability score request to an analytical management system 104, and present messages that it receives from the analytical management system 104 on a display (e.g., computer screen 103) and/or send (e.g., forward, redirect) the message to the one or more client device 102 to cause the one or more client devices 102 to present the message on a display of the one or more client devices 102.

The environment 100 includes a computer screen 103 (e.g., a monitor, a smartphone display) that is communicably coupled to the analytical management system104 for displaying information (e.g., a risk score, an impactability score).

Although FIG. 1 shows only a select number of computing devices (e.g., analytical management system 104, predictive model servers 107, notification system 106, client device 102, computer screen 103) and predictive model engines (e.g., risk predictive model engine, impactability predictive model engine), the environment 100 may include any number of computing devices (and predictive model engines) that are interconnected in any arrangement to facilitate the exchange of data between the computing devices.

The notification system 106 may send one or more requests (shown in FIG. 1 as “patient score requests”) to the analytical management system 104 for a risk score associated with a patient and/or a request for an impactability score associated with the patient. In response to receiving the request, the analytical management system 104 may determine if a predictive model engine is available to process the request. For example, if the request is for a risk score associated with the patient, then the analytical management system 104 determines if a risk predictive model engine 108a is available (e.g., deployed and/or idle) on the analytical management system 104 and/or the predictive model server 107 to process the request. If the request is for an impactability score associated with the patient, then the analytical management system 104 determines if an impactability predictive model engine 108b is available on the analytical management system 104 and/or the predictive model server 107 to process the request. If a predictive model engine is not available to process the request, then the analytical management system 104 may deploy one or more predictive model engines (e.g., a risk predictive model engine and/or an impactability predictive model engine) into the environment 100 according to the operations of the Training Phase, as discussed herein.

In response to receiving the request, the analytical management system 104 may generate one or more sets of scoring data that are associated with the patient (e.g., the patient that was identified in the request) based on data that the analytical management system 104 retrieves from database system 112. For example, if the request is for a risk score associated with the patient, then the analytical management system 104 generates a set of scoring data (sometimes referred to as “first set of scoring data”) that includes medical data associated with the patient, medical image scores associated with the patient, SDH scores associated with the patient, and/or clinician linkages associated with the patient. If the request is for an impactability score associated with the patient, then the analytical management system 104 generates a set of scoring data (sometimes referred to as “second set of scoring data”) that includes one or more subsets (or all subsets) of the first set of scoring data associated with the patient.

If the request is for a risk score associated with the patient, then the analytical management system 104 applies the first set of scoring data to a risk predictive model engine 108a. If the analytical management system 104 determines that a risk predictive model engine is executing on the analytical management system 104, then the analytical management system 104 applies the first set of scoring data to the risk predictive model engine 108a by sending a message (not shown in FIG. 1) to the risk predictive model engine 108a executing on the analytical management system 104. If the analytical management system 104 determines that a risk predictive model engine is executing on the predictive model server 107a, then the analytical management system 104 applies the first set of scoring data to the risk predictive model engine 108a by sending a message (shown in FIG. 1 as “AMS message”) to the predictive model server 107a, which in turn, sends (e.g., redirects, forwards) the message to the risk predictive model engine 108a executing on the predictive model server 107a.

If the request is for an impactability score associated with the patient, then the analytical management system 104 applies the second set of scoring data to an impactability predictive model engine 108b. If the analytical management system 104 determines that an impactability predictive model engine is executing on the analytical management system 104, then the analytical management system 104 applies the second set of scoring data to the impactability predictive model engine 108b by sending a message (not shown in FIG. 1) to the impactability predictive model engine 108b executing on the analytical management system 104. If the analytical management system 104 determines that an impactability predictive model engine is executing on the predictive model server 107b, then the analytical management system 104 applies the second set of scoring data to the impactability predictive model engine 108b by sending a message (shown in FIG. 1 as “AMS message”) to the predictive model server 107b, which in turn, sends (e.g., redirects, forwards) the message to the impactability predictive model engine 108b executing on the predictive model server 107b.

Applying the scoring data to the predictive model engine causes the predictive model engine to generate an output prediction based on the scoring data. For example, in response to receiving the first set of scoring data from the analytical management system 104, the risk predictive model engine 108a generates a risk score (e.g., an output prediction) based on the first set of scoring data, and the risk predictive model engine 108a and/or the predictive model server 107a send the risk score (shown in FIG. 1 as “risk score”) to the analytical management system 104. As another example, in response to receiving the second set of scoring data from the analytical management system 104, the impactability predictive model engine 108b generates an impactability score (e.g., an output prediction) based on the second set of scoring data, and the predictive model engine 108b and/or the predictive model server 107b to send the impactability score (shown in FIG. 1 as “impactability score”) to the analytical management system 104.

In response to receiving the risk score and/or the impactability score from the impactability predictive model engine 108b, the analytical management system 104 may send a message (sometimes referred to as an, “AMS message”) to the notification system, where the messages causes the notification system to present the risk score and/or the impactability score on a display of the notification system. The message may cause the notification system to send a second message (sometimes referred to as a “notification message”) to a client device to cause the client device to present the risk score and/or the impactability score on a display of the client device.

In response to receiving the risk score from the risk predictive model engine 108a, the analytical management system 104 may determine whether the risk score satisfies a criteria by comparing the risk score against the criteria. The analytical management system 104 may determine that the patient has a low probability of accessing in-person medical care at the medical provider within a predetermined temporal window by comparing the risk score to a criteria (e.g., a predetermined threshold value) and determining that determining that the risk score is less than the criteria. The analytical management system 104 may determine that the patient has a high probability of accessing in-person medical care at the medical provider within a predetermined temporal window by comparing the risk score to a criteria (e.g., a predetermined threshold value) and determining that determining that the risk score is greater than the criteria.

If the analytical management system 104 determines that the patient has a low probability of accessing in-person medical care at the medical provider within a predetermined temporal window, then the analytical management system 104 may determine to not send the scoring data (e.g., second set of scoring data) to the impactability predictive model engine 108b.

If the analytical management system 104 determines that the patient has a high probability of accessing in-person medical care at the medical provider within a predetermined temporal window, then the analytical management system 104 may proceed to determine whether an impactability predictive model engine 108b is available to process a request. If the analytical management system 104 determines that an impactability predictive model engine 108b is not available to process the request, then the analytical management system 104 may deploy an impactability predictive model engine 108b into the environment 100 according to the operations of the Training Phase, and proceed to apply the second set of scoring data to the impactability predictive model engine 108b. If the analytical management system 104 determines that an impactability predictive model engine 108b is available to process the request, then the analytical management system 104 may proceed to apply the second set of scoring data to the impactability predictive model engine 108b, which causes the impactability predictive model engine 108b to generate an impactability score based on the second set of scoring data, and the predictive model engine 108b and/or the predictive model server 107b to send the impactability score (shown in FIG. 1 as “impactability score”) to the analytical management system 104. That is, the analytical management system 104 may still provide an impactability score to the notification system 106 in the instances when the notification system 106 only sends a request (e.g., patient score request) for a risk score, if the analytical management system 104 determines that the risk score indicates that the patient has a high probability of accessing in-person medical care at the medical provider within a predetermined temporal window.

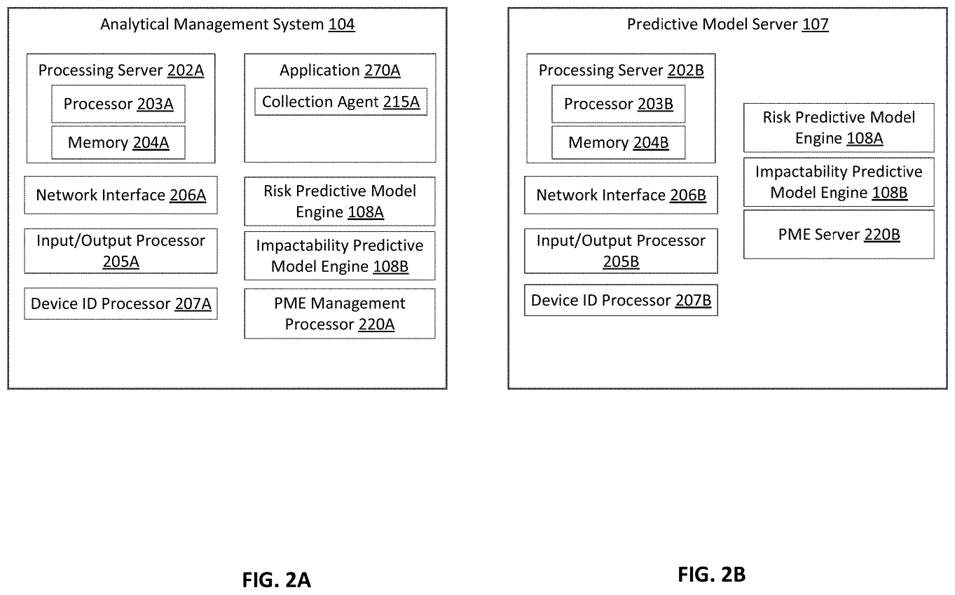

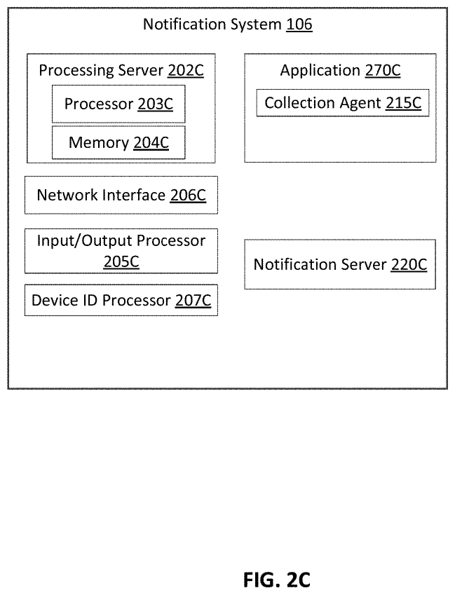

FIG. 2A is a block diagram depicting an example analytical management system (e.g., the environment in FIG. 1), according to some embodiments. While various servers, interfaces, and logic with particular functionality are shown, it should be understood that the analytical management system 104 includes any number of processors, servers, interfaces, and logic for facilitating the functions described herein. For example, the activities of multiple servers may be combined as a single server and implemented on a single processing server (e.g., processing server 202A), as additional servers with additional functionality are included.

The analytical management system 104 includes a processing server 202A composed of one or more processors 203A and a memory 204A. A processor 203A may be implemented as a general-purpose processor, a microprocessor, an Application Specific Integrated Circuit (ASIC), one or more Field Programmable Gate Arrays (FPGAs), a Digital Signal Processor (DSP), a group of processing components, or other suitable electronic processing components. In many embodiments, processor 203A may be a multi-core processor or an array (e.g., one or more) of processors.

The memory 104A (e.g., Random Access Memory (RAM), Read-Only Memory (ROM), Non-volatile RAM (NVRAM), Flash Memory, hard disk storage, optical media) of processing server 202A stores data and/or computer instructions/code for facilitating at least some of the various processes described herein. The memory 204A includes tangible, non-transient volatile memory, or non-volatile memory. The memory 204A stores programming logic (e.g., instructions/code) that, when executed by the processor 203A, controls the operations of the analytical management system 104. In some embodiments, the processor 203A and the memory 204A form various processing servers described with respect to the analytical management system 104. The instructions include code from any suitable computer programming language such as, but not limited to, C, C++, C#, Java, JavaScript, VBScript, Perl, HTML, XML, Python, TCL, and Basic. In some embodiments (referred to as “headless servers”), the analytical management system 104 may omit the input/output processor (e.g., input/output processor 205A), but may communicate with an electronic computing device via a network interface (e.g., network interface 206A).

The analytical management system 104 includes a network interface 206A configured to establish a communication session with a computing device for sending and receiving data over a network to the computing device. Accordingly, the network interface 206A includes a cellular transceiver (supporting cellular standards), a local wireless network transceiver (supporting 802.11X, ZigBee, Bluetooth, Wi-Fi, or the like), a wired network interface, a combination thereof (e.g., both a cellular transceiver and a Bluetooth transceiver), and/or the like. In some embodiments, the analytical management system 104 includes a plurality of network interfaces 206A of different types, allowing for connections to a variety of networks, such as local area networks or wide area networks including the Internet, via different sub-networks.

The analytical management system 104 includes an input/output server 205A configured to receive user input from and provide information (e.g., patient score requests, AMS messages, risk scores, impactability scores, notifications, alerts) to a user of the analytical management system 104. In this regard, the input/output processor 205A is structured to exchange data, communications, instructions, etc. with an input/output component of the analytical management system 104. Accordingly, input/output processor 205A may be any electronic device that conveys data to a user by generating sensory information (e.g., a visualization on a display, one or more sounds, tactile feedback) and/or converts received sensory information from a user into electronic signals (e.g., a keyboard, a mouse, a pointing device, a touch screen display, a microphone). The one or more user interfaces may be internal to the housing of the analytical management system 104, such as a built-in display, touch screen, microphone, etc., or external to the housing of the analytical management system 104, such as a monitor connected to the analytical management system 104, a speaker connected to the analytical management system 104, etc., according to various embodiments. In some embodiments, the input/output processor 205A includes communication processors, servers, and circuitry for facilitating the exchange of data, values, messages (e.g., patient score requests, AMS messages, risk scores, impactability scores), and the like between the input/output device and the components of the analytical management system 104. In some embodiments, the input/output processor 205A includes machine-readable media for facilitating the exchange of information between the input/output device and the components of the analytical management system 104. In still another embodiment, the input/output processor 205A includes any combination of hardware components (e.g., a touchscreen), communication processors, servers, circuitry, and machine-readable media.

The analytical management system 104 includes a device identification processor 207A (shown in FIG. 2A as device ID processor 207A) configured to generate and/or manage a device identifier (e.g., a media access control (MAC) address, an internet protocol (IP) address) associated with the analytical management system 104. The device identifier may include any type and form of identification used to distinguish the analytical management system 104 from other computing devices. To preserve privacy, the device identifier may be cryptographically generated, encrypted, or otherwise obfuscated by any server/processor of the analytical management system 104. The analytical management system 104 may include the device identifier in any communication (any of the messages in FIG. 1, e.g., patient score requests, AMS messages, risk scores, impactability scores) that the analytical management system 104 sends to a computing device.

The analytical management system 104 includes (or executes) an application 270A that the analytical management system 104 displays on a computer screen (local or remote) allowing a user of the analytical management system 104 to view and exchange data (e.g., patient score requests, AMS messages, risk scores, impactability scores) with any other computing devices (e.g., client device 102, predictive model servers 107, database system 112) connected to the communication network 120, or any processor/server and/or subsystem (e.g., risk predictive model engine 108a, impactability predictive model engine 108B, predictive model engine (PME) management processor 220A) of the analytical management system 104.

The application 270A includes a collection agent 215A. The collection agent 215A may include an application plug-in, application extension, subroutine, browser toolbar, daemon, or other executable logic for collecting data processed by the application 270A and/or monitoring interactions of user with the input/output processor 205A. In other embodiments, the collection agent 215A may be a separate application, service, daemon, routine, or other executable logic separate from the application 270A but configured for intercepting and/or collecting data processed by application 270A, such as a screen scraper, packet interceptor, application programming interface (API) hooking process, or other such application. The collection agent 215A is configured for intercepting or receiving data input via the input/output processor 205A, including mouse clicks, scroll wheel movements, gestures such as swipes, pinches, or touches, or any other such interactions; as well as data received and processed by the application 270A. The collection agent 215A, may begin intercepting/gathering/receiving data input via its respective input/output processor based on any triggering event, including, e.g., a power-up of the analytical management system 104 or a launch of any software application executing on processing server 202A.

The analytical management system 104 may optionally include a risk predictive model engine 108A and/or an impactability predictive model engine 108B that each execute (e.g., run) on the processor 203A of the analytical management system 104.

The analytical management system 104 includes a predictive model engine (PME) management processor 220A that may be configured to receive a request (shown in FIG. 1 as “patient score request”) for one or more scores (e.g., a risk score, an impactability score) associated with a patient of one or more medical providers. The PME management processor 220A may be configured to extract information (e.g., a patient identifier, a description of a predetermined temporal window) from the request in response to receiving the request.

The PME management processor 220A may be configured to retrieve one or more sets of data from database system 112. The PME management processor 220A may be configured to generate a one or more sets of scoring data that are associated with the patient (e.g., the patient that was identified in the request) based on data that the PME management processor 220A retrieves from a database.

The PME management processor 220A may be configured to determine whether a predictive model engine 108 (e.g., a risk predicative model engine, an impactability predictive model engine) is available to process a request. The PME management processor 220A may be configured to, deploy one or more predictive model engines 108 into an environment (e.g., environment 100 in FIG. 1) according to the operations of the Training Phase, as discussed herein, if the PME management processor 220A determines that an impactability predictive model engine 108B is not available to process the request.

The PME management processor 220A may train any of the predictive model engines 108 depicted in FIG. 1 (e.g., any predictive model engine executing on analytical management system 104 and/or on predictive model servers 107) according to the operations of the Training Phase, as discussed herein.

The PME management processor 220A may be configured to apply one or more sets of scoring data associated with the patient to a predictive model engine 108 (e.g., a risk predictive model engine) that is trained with training data to cause the predictive model engine 108 to generate a score (e.g., a risk score) associated with a first predetermined temporal window and based on the one or more sets of scoring data, and send the score to the PME management processor 220A. In some embodiments, the first predetermined temporal window may be based on a default time (stored in a memory or database) instead of a predetermined temporal window that was included (or referenced) in the request.

The PME management processor 220A may be configured to receive one or more scores score from a predictive model engine 108 and/or a predictive model server 107.

The PME management processor 220A may be configured to determine that a score (e.g., a risk score) satisfies a criteria. The PME management processor 220A may be configured to determine that the score satisfies a criteria by comparing the score to the criteria.

The PME management processor 220A may be configured to apply, in response to determining that the score satisfies the criteria, one or more sets of scoring data associated with the patient to another predictive model engine 108 (e.g., an impactability predictive model engine) that is trained with training data to cause the predictive model engine 108B to generate a score (e.g., an impactability score) associated with a second predetermined temporal window and based on the one or more sets of scoring data. In some embodiments, the second predetermined temporal window may be based on a default time (stored in a memory or database) instead of a predetermined temporal window that was included (or referenced) in the request. In some embodiments, the first predetermined temporal window and the second predetermined temporal window may be the same predetermined temporal window or different predetermined temporal windows.

The PME management processor 220A may be configured to send a message (sometimes referred to as an, “AMS message”) to a notification system 106, where the messages causes the notification system to present a risk score and/or an impactability score on a display of the notification system 106. In some embodiments, the message may cause the notification system 106 to send a second message (sometimes referred to as a “notification message”) to a one or more client devices 102 to cause the one or more client devices 102 to present the risk score and/or the impactability score on a display of the one or more client devices 102.

The analytical management system 104 includes a bus (not shown), such as an address/data bus or other communication mechanism for communicating information, which interconnects processors, servers, and/or subsystems of the analytical management system 104. In some embodiments, the analytical management system 104 may include one or more of any such processors, servers, and/or subsystems.

In some embodiments, some or all of the processors/servers of the analytical management system 104 may be implemented with the processing server 202A. For example, any of the analytical management system 104 may be implemented as a software application stored within the memory 204A and executed by the processor 203A. Accordingly, such arrangement can be implemented with minimal or no additional hardware costs. Any of these above-recited servers/processors may rely on dedicated hardware specifically configured for performing operations of the server/processor.

FIG. 2B is a block diagram depicting an example predictive model server (e.g., the environment in FIG. 1), according to some embodiments. While various processors, servers, interfaces, and logic with particular functionality are shown, it should be understood that the predictive model server 107 includes any number of processors, servers, interfaces, and logic for facilitating the functions described herein. For example, the activities of multiple processors and/or servers may be combined as a single server and/or processor and implemented on a single processing server (e.g., processing server 202B), as additional processors/servers with additional functionality are included.

The predictive model server 107 includes a processing server 202B composed of one or more processors 203B and a memory 204B. The processing server 202B includes identical or nearly identical functionality as processing server 202A in FIG. 2A, but with respect to processors, servers, and/or subsystems of the predictive model server 107 instead of processors, servers, and/or subsystems of the analytical management system 104.

The memory 204B of processing server 202B stores data and/or computer instructions/code for facilitating at least some of the various processes described herein. The memory 204B includes identical or nearly identical functionality as memory 204A in FIG. 2A, but with respect to processors, servers, and/or subsystems of the predictive model server 107 instead of processors, servers, and/or subsystems of the analytical management system 104.

The predictive model server 107 includes a network interface 206B configured to establish a communication session with a computing device for sending and receiving data over a network to the computing device. Accordingly, the network interface 206B includes identical or nearly identical functionality as network interface 206A in FIG. 2A, but with respect to processors, servers, and/or subsystems of the predictive model server 107 instead of processors, servers, and/or subsystems of the analytical management system 104.

The predictive model server 107 includes an input/output processor 205B configured to receive user input from and provide information to a user. In this regard, the input/output processor 205B is structured to exchange data, communications, instructions, etc. with an input/output component of the predictive model server 107. The input/output processor 205B includes identical or nearly identical functionality as input/output processor 205A in FIG. 2A, but with respect to processors, servers, and/or subsystems of the predictive model server 107 instead of processors, servers, and/or subsystems of the analytical management system 104.

The predictive model server 107 includes a device identification processor 207B (shown in FIG. 2B as device ID processor 207B) configured to generate and/or manage a device identifier associated with the predictive model server 107. The device ID processor 207B includes identical or nearly identical functionality as device ID processor 207A in FIG. 2A, but with respect to processors, servers, and/or subsystems of the predictive model server 107 instead of processors, servers, and/or subsystems of the analytical management system 104.

The predictive model server 107 includes one or more predictive model engines (e.g., a risk predictive model engine 108A, an impactability predictive model engine 108B) that each execute on the processor 203B of the predictive model server 107.

The predictive model server 107 includes a predictive model engine (PME) server 220B that may be configured to receive a set of scoring data from the analytical management system 104. The PME server 220B may be configured to select the risk predictive model engine 108A or the impactability predictive model engine 108B to process the scoring data based on the set of scoring data, and apply (e.g., insert, provide) the scoring data to the selected predictive model engine to cause the selected predictive model engine to generate a score (e.g., a risk score, an impactability score). The PME server 220B may be configured to send the score to the analytical management system 104.

The predictive model server 107 includes a bus (not shown), such as an address/data bus or other communication mechanism for communicating information, which interconnects processors, servers, and/or subsystems of the predictive model server 107. In some embodiments, the predictive model server 107 may include one or more of any such processors, servers, and/or subsystems.

In some embodiments, some or all of the processors and/or servers of the predictive model server 107 may be implemented with the processing server 202B. For example, any of the predictive model server 107 may be implemented as a software application stored within the memory 204B and executed by the processor 203B. Accordingly, such arrangement can be implemented with minimal or no additional hardware costs. Any of these above-recited processors/servers may rely on dedicated hardware specifically configured for performing operations of the processors/server.

FIG. 2C is a block diagram depicting an example notification system (e.g., the environment in FIG. 1), according to some embodiments. While various processors, servers, interfaces, and logic with particular functionality are shown, it should be understood that the notification system 106 includes any number of servers, interfaces, and logic for facilitating the functions described herein. For example, the activities of multiple servers may be combined as a single server and implemented on a single processing server (e.g., processing server 202C), as additional servers with additional functionality are included.

The notification system 106 includes a processing server 202C composed of one or more processors 203C and a memory 204C. The processing server 202C includes identical or nearly identical functionality as processing server 202A in FIG. 2A, but with respect to servers and/or subsystems of the notification system 106 instead of servers and/or subsystems of the analytical management system 104.

The memory 204C of processing server 202C stores data and/or computer instructions/code for facilitating at least some of the various processes described herein. The memory 204C includes identical or nearly identical functionality as memory 204A in FIG. 2A, but with respect to servers and/or subsystems of the notification system 106 instead of servers and/or subsystems of the analytical management system 104.

The notification system 106 includes a network interface 206C configured to establish a communication session with a computing device for sending and receiving data over a network to the computing device. Accordingly, the network interface 206C includes identical or nearly identical functionality as network interface 206A in FIG. 2A, but with respect to servers and/or subsystems of the notification system 106 instead of servers and/or subsystems of the analytical management system 104.

The notification system 106 includes an input/output server 205B configured to receive user input from and provide information to a user. In this regard, the input/output server 205B is structured to exchange data, communications, instructions, etc. with an input/output component of the predictive model server 107. The input/output server 205B includes identical or nearly identical functionality as input/output processor 205A in FIG. 2A, but with respect to servers and/or subsystems of the notification system 106 instead of servers and/or subsystems of the analytical management system 104.

The notification system 106 includes a device identification server 207C (shown in FIG. 2C as device ID server 207C) configured to generate and/or manage a device identifier associated with the notification system 106. The device ID server 207C includes identical or nearly identical functionality as device ID processor 207A in FIG. 2A, but with respect to servers and/or subsystems of the notification system 106 instead of servers and/or subsystems of the analytical management system 104.

The notification system 106 includes (or executes) an application 270C that the notification system 106 displays on a computer screen allowing a user of the notification system 106 to view and exchange data (e.g., patient score requests, AMS messages, risk scores, impactability scores) with any other computing devices (e.g., client device 102, analytical management system 104) connected to the communication network 120, or any server and/or subsystem of the notification system 106. The application 270C includes a collection agent 215C. The application 270C and the collection agent 215C include identical or nearly identical functionality as their respective counter-part (e.g., application 270A in FIG. 2A and collection agent 215A in FIG. 1A), but with respect to servers and/or subsystems of the notification system 106 instead of servers and/or subsystems of the analytical management system 104.

The notification system 106 includes a notification server 220C that may be configured to configured to generate and send a request (shown in FIG. 1 as “patient score request”) to an analytical management system 104 for one or more scores (e.g., a risk score, an impactability score) associated with a patient of one or more medical providers.

The notification server 220C may be configured to receive a message (sometimes referred to as an, “AMS message”) from the analytical management system 104. The notification server 220C may be configured to exact a risk score and/or an impactability score from the message and present the extracted scores on a display of the notification system 106. The notification server 220C may be configured to send (e.g., forward, redirect) the message to one or more client devices 102, causing the one or more client devices 102 to present a risk score and/or an impactability score on a display of the one or more client devices 102.

The notification system 106 includes a bus (not shown), such as an address/data bus or other communication mechanism for communicating information, which interconnects servers and/or subsystems of the predictive model server 107. In some embodiments, the notification system 106 may include one or more of any such servers and/or subsystems.

In some embodiments, some or all of the servers of the notification system 106 may be implemented with the processing server 202C. For example, any of the notification system 106 may be implemented as a software application stored within the memory 204C and executed by the processor 203C. Accordingly, such arrangement can be implemented with minimal or no additional hardware costs. Any of these above-recited servers may rely on dedicated hardware specifically configured for performing operations of the server.

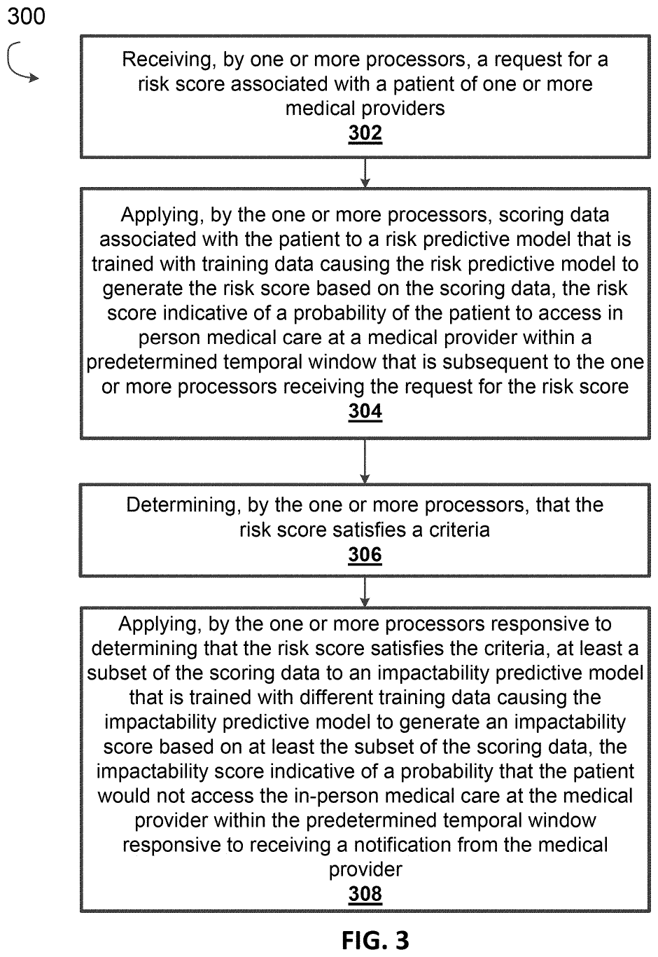

FIG. 3 is a flow diagram depicting a method for predicting and improving the healthcare decisions of a patient via predictive modeling, according to some embodiments. Additional, fewer, or different operations may be performed in the method depending on the particular arrangement. In some arrangements, some or all operations of method 300 may be performed by one or more processors executing on one or more computing devices, systems, or servers. In some arrangements, some or all operations of method 300 may be performed by one or more analytical management systems, such as analytical management system 104 in FIG. 1. In some arrangements, some or all operations of method 300 may be performed by one or more predictive model servers, such as predictive model server 107 in FIG. 1. In some arrangements, some or all operations of method 300 may be performed by one or more notification systems, such as notification system 106 in FIG. 1. Each operation may be re-ordered, added, removed, or repeated.

As shown in FIG. 3, illustrates a flowchart depicting operational steps for generating and executing the artificial intelligence (AI) models (e.g., risk model and the impactability predictive model) described herein. Embodiments of executing the method 300 may comprise additional or alternative steps, or may omit some steps altogether. Even though certain steps described herein are described as being executed by a central server, the steps can be performed by different processors and/or servers described herein. For instance, the server performing the method 300 may refer to the servers/processors described in FIGS. 1-2C (e.g., servers and/or processors of the analytical management system, the predictive model server, and/or the notification system). In some embodiments, a single server may act as all the above-described systems, such that a single server generates/executes the AI models and displays the results on client computing devices. In some embodiments, one or more steps of the method 300 may be performed by each system server described herein.

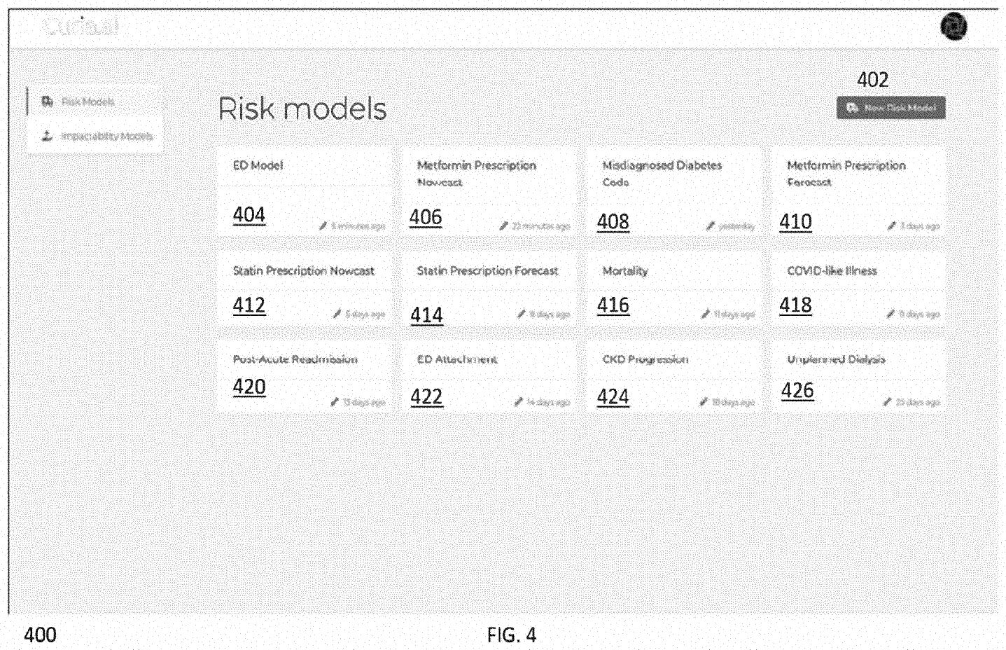

The method 300 includes step 302 of receiving, by one or more processors, a request for a risk score associated with a patient of one or more medical providers. As described herein, one or more processors/servers, may receive a request from a client device (e.g., client device 102 in FIG. 1) operated by an end-user to generate the described risk and impactability predictive models. The request may also include various attributes associated with each predictive model, such as patient classifications, procedures to be predicted, timelines, data to be analyzed, and the like. For instance, the end-user may access various graphical user interfaces hosted or otherwise generated by the server to input his/her request, as depicted in FIGS. 4-9.

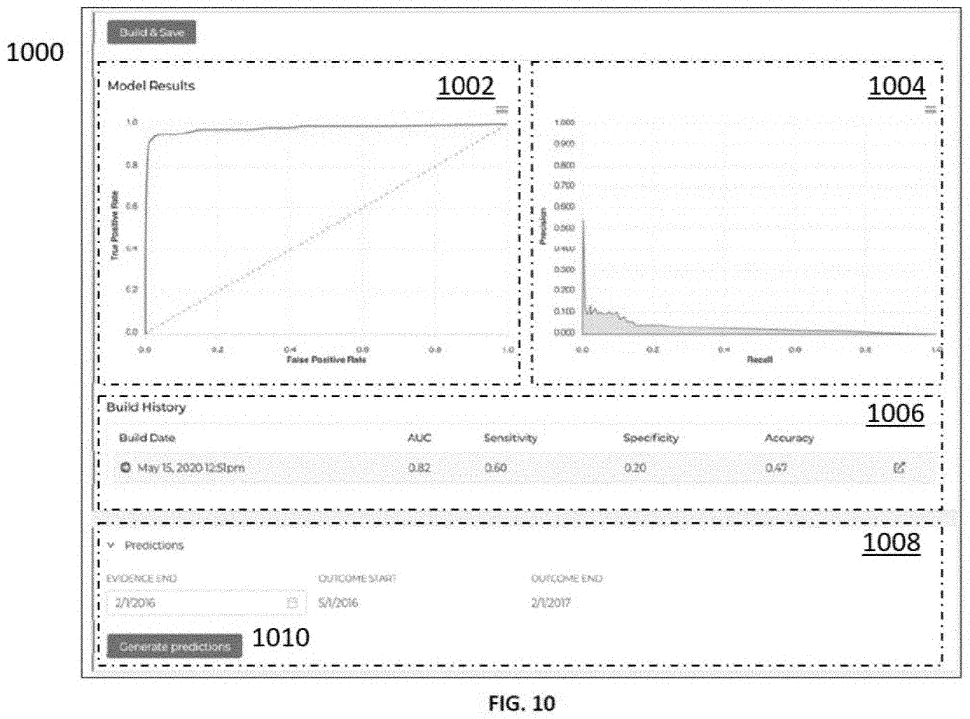

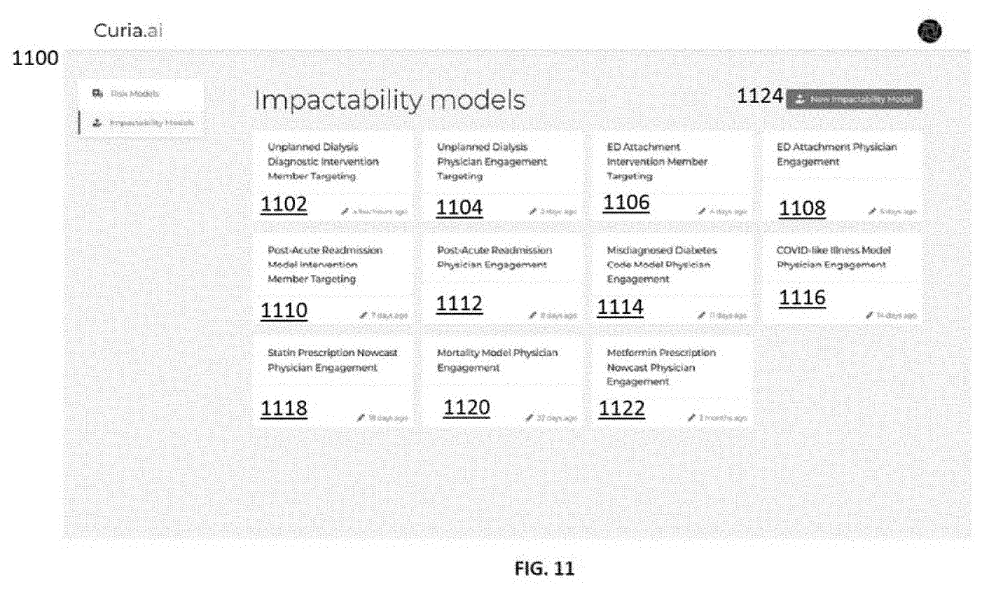

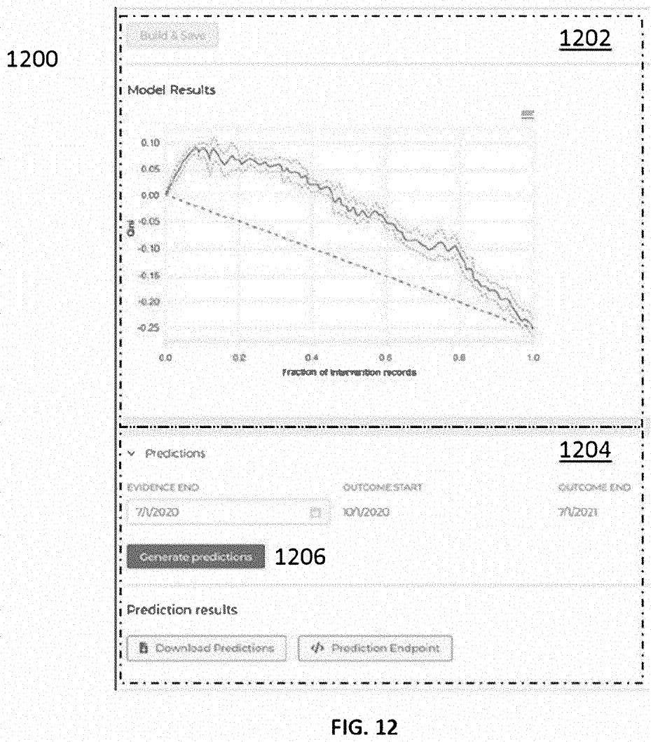

Referring now to FIGS. 4-12 various graphical user interfaces provided by one or more servers of one or more systems described herein, such as system 100 described in FIG. 1 are depicted. Using various graphical components provided within the GUIs described herein, an end-user may select various attributes used by one or more servers described herein to generate and execute the risk and/or impactability predictive models, such that the results are applicable to the end-user's particular needs. For instance, using the graphical components described herein a user may tailor the results of the risk and/or impactability predictive model by instructing one or more servers to analyze a particular group of attributes or patients. In this example, the user experience may start by an end-user interacting with the GUI depicted in FIG. 4 and may end by the user viewing the results displayed in FIGS. 10 and 12.

FIG. 4 is a graphical user interface of an example application depicting a method for displaying a plurality of risk models, according to some embodiments. The application may be application 270A in FIG. 2A that executes on the analytical management system 104. The application may be application 270C in FIG. 2C that executes on the notification system 106. The end-user may use various graphical components displayed to generate the risk model. For instance, the end-user may be a healthcare professional who would like to identify a list of patients who are at a high risk of returning for additional medical procedures.

The end-user may interact with the button 402 and instruct the one or more processors (e.g., one or more processors of the analytical management system described in FIG. 2A) to create a risk model. Upon receiving the end-user's request, the GUI 400 may further display various graphical components 404-426. For instance, the end-user may limit the risk factor to the risk of patients visiting a medical facility for an unplanned dialysis (graphical component 426) or COVID-like illnesses (graphical component 418). The end-user may select one or more attributes to be included in the risk model. Upon receiving the end-user's selections, one or more processors described herein (e.g., processors of the analytical management system 104) may generate the risk model using the methods and systems described herein.

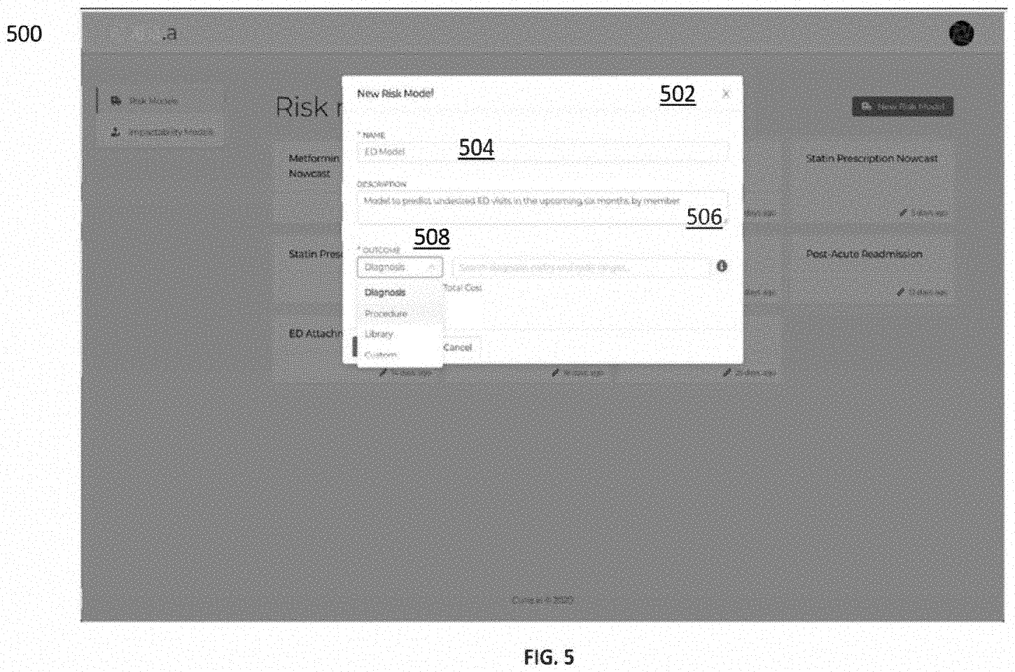

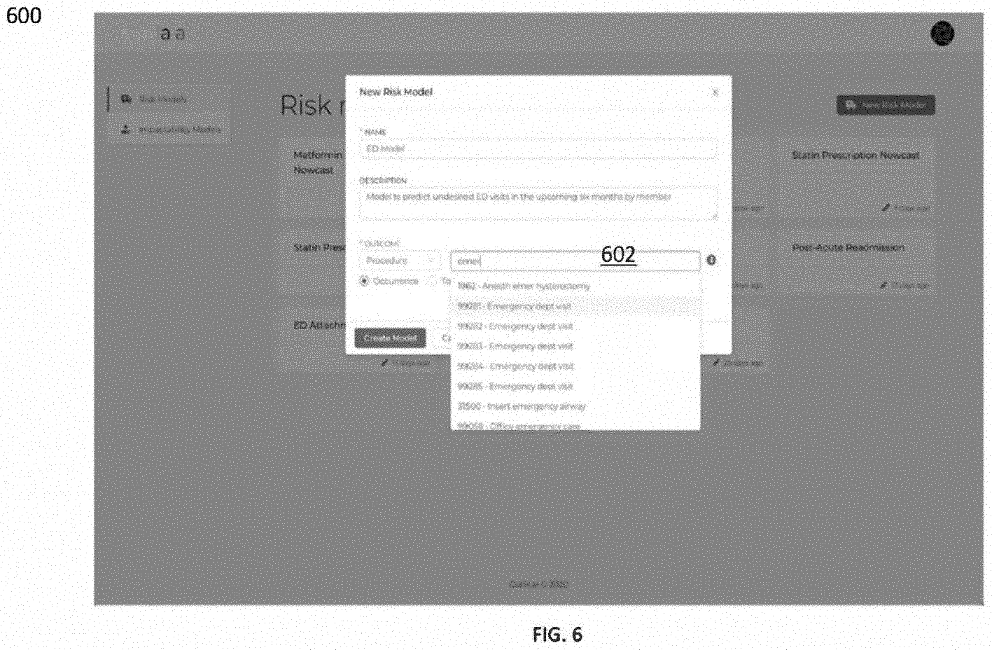

FIG. 5 is a graphical user interface of an example application depicting a method for selecting an outcome for a risk model, according to some embodiments. The application may be application 270A in FIG. 2A that executes on the analytical management system 104. The application may be application 270C in FIG. 2C that executes on the notification system 106. As described above, the end-user may customize the risk model, such that the results indicate a likelihood of a particular patient visiting a doctor for a specific reason.

In the non-limiting example depicted herein, when the end-user selects emergency department visits (ED visit) risk model, one or more processors described herein display the GUI 500 that include the graphical component 502. The graphical component 502 allows the user to choose a name for the newly generated risk model (component 504) and provide a brief description of the model (component 506). The GUI 500 may also include the drop-down component 508 that indicates the outcome desired by the end-user. For instance, the end-user may customize the risk model such that it only applies to patient visits based on a particular diagnosis (e.g., patients visiting the emergency department because of or after a particular diagnosis) or a particular procedure (e.g., ED visits because of or after a particular procedure). For instance, when the end-user selects “procedure,” the risk model may identify a likelihood of the patient revisiting the emergency department for a medical code associated with one or more procedures inputted by the end-user. The drop down 508 may also include a “library” feature, which provides pre-constructed attributes to be selected by the end-user.

FIG. 6 is a graphical user interface of an example application depicting a method for searching procedure codes and code ranges for a risk model, according to some embodiments. The application may be application 270A in FIG. 2A that executes on the analytical management system 104. The application may be application 270C in FIG. 2C that executes on the notification system 106.

When the end-user selects “procedure” from the drop-down menu 508, one or more processors described herein are displayed on the GUI 600. The graphical component 602 allows the end-user to enter and/or select a particular procedure. As described above, selecting “procedure” results in a risk model that identifies a likelihood of one or more patients visiting the emergency department for reasons that are associated with a particular procedure (e.g. inputted by the end-user into the input field 602). The end-user may also tailor the risk model, such that the risk model identifies a likelihood of patients visiting the emergency department for all procedures.

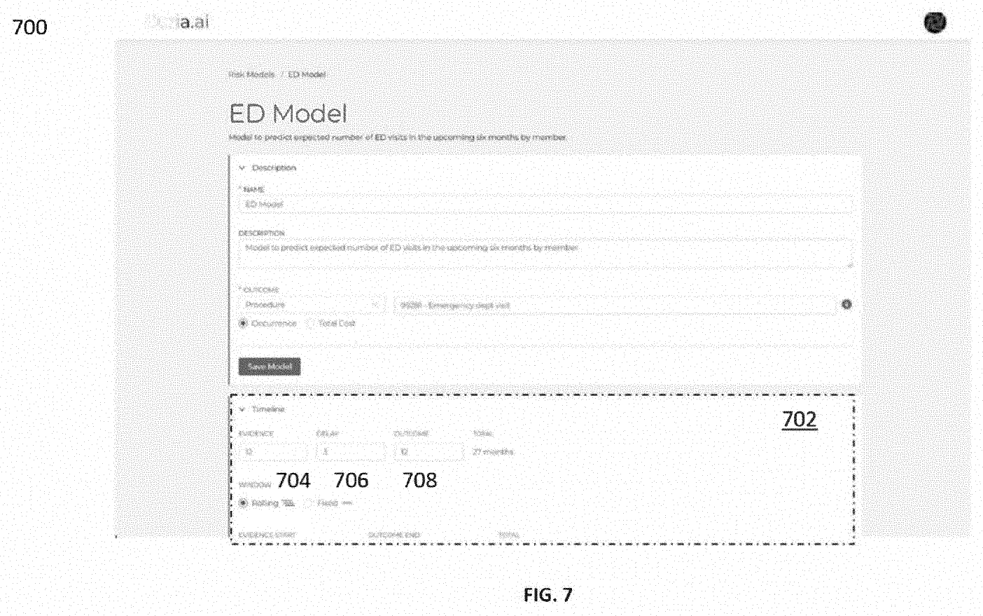

FIG. 7 is a graphical user interface of an example application depicting a method for selecting a predetermined temporal window as a condition for executing a risk model, according to some embodiments. The application may be application 270A in FIG. 2A that executes on the analytical management system 104. The application may be application 270C in FIG. 2C that executes on the notification system 106.

The end-user may also tailor the timeline associated with the risk model. For instance, as depicted in GUI 700, the end-user may enter a time for the potential visit of each patient. For instance, the end-user may use various input elements illustrated in the graphical component 702 (e.g., text string input elements and/or drop-down menus) to enter a particular time window. As a result, the risk model calculates a likelihood of the patient's visit to the emergency department for a particular procedure (inputted by the end-user, as illustrated in FIG. 6) using patient data within the identified window. In the non-limiting example depicted in FIG. 7, the user desires the risk model that identifies a likelihood of patients visiting the emergency department for any procedure using patient data for the previous 27 months.

The timeline inputted by the end-user may correspond to a rolling or fixed window of time. Using the “fixed” option, the end-user can define an overall time window and instruct the risk model to calculate the likelihoods accordingly. For instance, using this option, the end-user may instruct the risk model to use all patient data for the past 12 months. Using the “rolling” option, the end-user can define the time window, such that a specific amount of time is excluded from the risk model's analysis. For instance, as depicted in the graphical component 702, the end-user indicates that he/she would like the risk model to use patient data within the last 12 months from the current date (input field 704), exclude patient data from 13-15 months from the current date (706), and include patient data corresponding to the prior 12 months (past 16-27 months, as entered in the input element 708). Defining specific time windows allows the end-user to avoid peculiar/outlier data (e.g., patient information during a pandemic that may negatively affect the risk model, such as the COVID-19 pandemic in 2020).

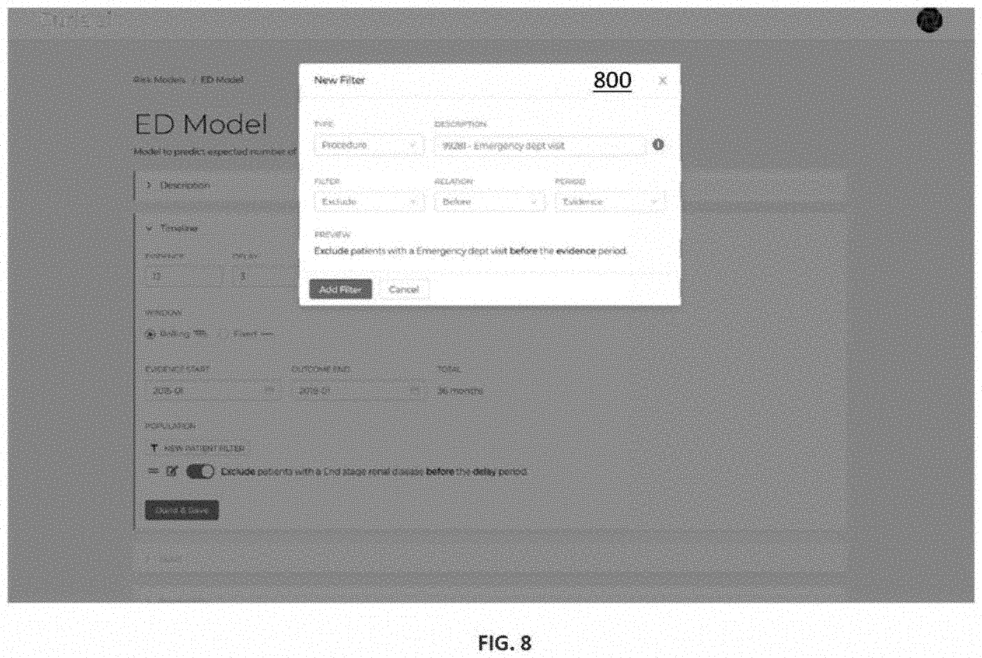

FIG. 8 is a graphical user interface of an example application depicting a method for filtering training data and/or scoring data for executing a risk model, according to some embodiments. The application may be application 270A in FIG. 2A that executes on the analytical management system 104. The application may be application 270C in FIG. 2C that executes on the notification system 106. The graphical component 800 provides the end-user the options to create various filters, such that the risk model excludes data indicated by the end-user.

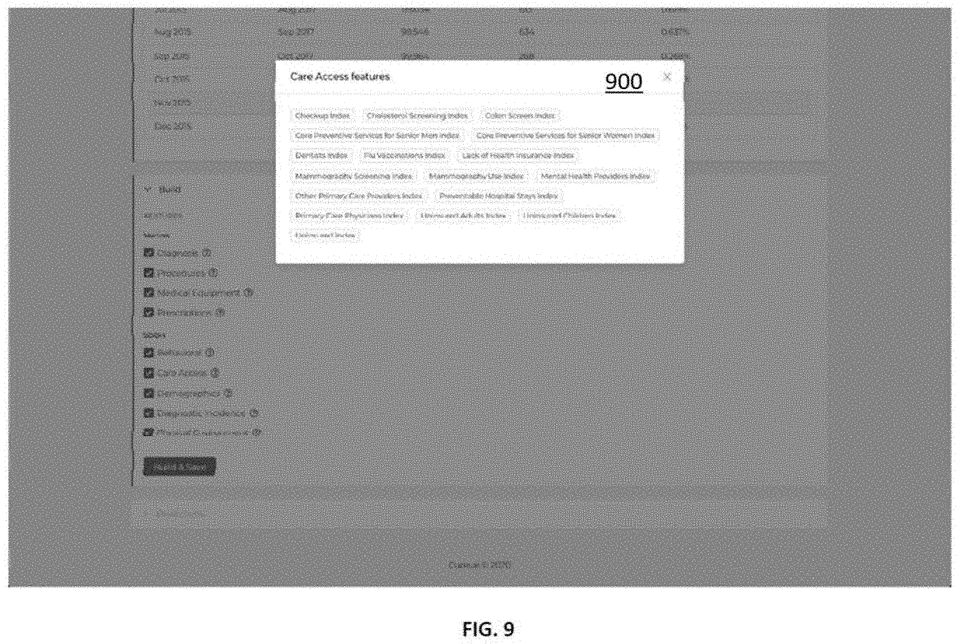

FIG. 9 is a graphical user interface of an example application depicting a method for displaying a plurality of care access features for a risk model, according to some embodiments. The application may be application 270A in FIG. 2A that executes on the analytical management system 104. The application may be application 270C in FIG. 2C that executes on the notification system 106. As another step of creating the risk model, the end-user may select the data to be considered when creating the risk model. For instance, as depicted in the graphical component 900, the end-user may select one or more normalized indexes corresponding to specific data collected and processed by one or more processors/servers of the analytical management system.

FIG. 10 is a graphical user interface of an example application depicting a method for displaying the output predictions and/or model results from a risk model, according to some embodiments. The application may be application 270A in FIG. 2A that executes on the analytical management system 104. The application may be application 270C in FIG. 2C that executes on the notification system 106.

One or more processors and servers, such as the predictive model server described in FIG. 2B may execute the generated/trained risk model. As will be described below, a secondary model (e.g., impactability predictive model) may ingest the results generated by the risk model. However, the predictive model server and/or any server/processor of the notification system may also display the results generated by the risk model. For instance, the end-user may view and/or revise various details associated with the risk model, as depicted in GUI 1000. More specifically, GUI 1000 may visually illustrate the analytical performance of the risk model.

The GUI 1000 may include chart 1002 that indicates true and false positive rates. For instance, the risk model may perform its predictive calculation based on previously known ground truth data (e.g., previous patients who have visited ED) and identify whether the results produced by the risk model corresponds to the data indicated within the ground truth dataset. The GUI 1000 may also include chart 1004 may correspond to precision/recall attributes of the data produced by the risk model.

The GUI 1000 may also include various measurements associated with the performance of the risk model. For instance, the graphical component 1006 identify a “build the history” of the risk model that has been generated based on user inputs and selections depicted in FIGS. 4-9. The graphical component 1006 may indicate a date associated with generation of the risk model and various analytical parameters (e.g., precision/recall AUC, sensitivity, specificity, and accuracy of the risk model). The end-user may review the graphical component 1006 along with the charts 1002 and 1004 to ensure that the risk model satisfies various predetermined performance thresholds. If so, the end-user may execute the risk model using the interactive button 1010 depicted in the graphical component 1008. For instance, and referring back to the risk model generated and describe in FIGS. 4-9, the end-user may execute the risk model to identify a likelihood of one or more patients visiting the emergency department.

FIG. 11 is a graphical user interface of an example application depicting a method for displaying a plurality of impactability predictive models, according to some embodiments. The application may be application 270A in FIG. 2A that executes on the analytical management system 104. The application may be application 270C in FIG. 2C that executes on the notification system 106. Similar to generating the risk model, depicted in FIG. 4, the end-user may select one or more attributes depicted in GUI 1100 (e.g., graphical components 1102-1122). The end-user can then generate the impactability predictive model by interacting with the button 1124.

Upon generation of the impactability predictive model, the end-user may also view FIG. 12, which is a graphical user interface of an example application depicting a method for displaying the output predictions and/or model results from an impactability model, according to some embodiments. The application may be application 270A in FIG. 2A that executes on the analytical management system 104. The application may be application 270C in FIG. 2C that executes on the notification system 106.

GUI 1200 includes the chart 1202 that displays the impactability predictive model results. The chart 1202 illustrates that the likelihood of patients visiting the emergency department (illustrated on the Y-axis) decreases as the number of interventions (illustrated in the X-axis) increases. Therefore, the chart 1202 illustrates that when certain patients are contacted by a medical professional, their likelihood of returning to the emergency department decreases. The end-user may then interact with the button 1206 and input various elements within the graphical component 1204 to execute the impactability predictive model for a selected set of patients and review the results.

Referring back to FIG. 3, the method 300 also includes step 304 of applying, by the one or more processors, scoring data associated with the patient to a risk predictive model that is trained with training data causing the risk predictive model to generate the risk score based on the scoring data, the risk score indicative of a probability of the patient to access in person medical care at a medical provider within a predetermined temporal window that is subsequent to the one or more processors receiving the request for the risk score.

Based on the request received from the user, the server may generate the risk and the impactability predictive model using various artificial intelligence modeling techniques described herein, such as neural networks, deep learning, and the like. The server may then execute the risk model to identify a likelihood of each patient (having been previously treated or associated with a medical procedure) needing further medical assistance. For instance, the server may execute the risk model by inputting attributes of current patients (e.g., each patient's physical attributes, a time associated with each patient's medical procedure previously conducted, and/or attributes of the medical procedure). As a result, the server may identify a likelihood of each patient needing further medical assistance (e.g., the medical assistance as defined by the end the user, such as visiting the emergency room or needing a particular medical procedure).

The method 300 also includes step 306 of determining, by the one or more processors, that the risk score satisfies a criteria. The server may then identify a subset of the patients who satisfy a predetermined criteria/threshold. The server may analyze the results achieved by executing the risk model (step 304) and may identify a list of patients whose likelihood of needing further medical assistance exceeds a predetermined threshold. For example, the server may identify every patient with an 80% (or higher) likelihood of visiting the emergency department. As described herein, the predetermined threshold may be inputted and/or revised by the end-user.

The method 300 also includes step 308 of applying, by the one or more processors responsive to determining that the risk score satisfies the criteria, at least a subset of the scoring data to an impactability predictive model that is trained with different training data causing the impactability predictive model to generate an impactability score based on at least the subset of the scoring data, the impactability score indicative of a probability that the patient would not access the in-person medical care at the medical provider within the predetermined temporal window responsive to receiving a notification from the medical provider.

As described herein, one or more processors/servers (e.g., the predictive model server) may also generate a secondary model (e.g., impactability predictive model) to ingest the data generated by the risk model. The end-user may generate the impactability predictive model using similar methods as described above (e.g., inputting various features and attributes using GUIs depicted in FIGS. 4-12).

The server may then execute the secondary model (e.g., impactability predictive model) for the subset of the patients identified in step 306. Therefore, the impactability predictive model may ingest the data generated via execution of the risk model to produce a likelihood of a patient being impacted, such that patient no longer needs further medical assistance (e.g., the patient no longer needs to visit the emergency department). The impactability predictive model may then display a list of patients who are more likely to be influenced after communicating with a medical professional. One or more servers/processors described herein can then automatically establish an electronic communication session between the end-user's client device and an electronic device of the patients. For instance, the end-user may interact with a particular patient's indicator (e.g., name) where the server establishes an electronic communication with the patient (e.g., calls the patients or initiates an email application executing on the end-user's client device).

The embodiments described herein have been described with reference to drawings. The drawings illustrate certain details of specific arrangements that implement the systems, methods and programs described herein. However, describing the arrangements with drawings should not be construed as imposing on the disclosure any limitations that may be present in the drawings.

As used herein, the terms “server” and/or “processor” may include hardware structured to execute the functions described herein. In some arrangements, each respective “server” and/or “processor” may include machine-readable media for configuring the hardware to execute the functions described herein. The server may be embodied as one or more circuitry components including, but not limited to, processing circuitry, network interfaces, peripheral devices, input devices, output devices, sensors, etc. In this regard, the “server” or “processor” may include any type of component for accomplishing or facilitating achievement of the operations described herein.

The “server” may also include one or more processors communicatively coupled to one or more memory or memory devices. In this regard, the one or more processors may execute instructions stored in the memory or may execute instructions otherwise accessible to the one or more processors. In some arrangements, the one or more processors may be embodied in various ways. The one or more processors may be constructed in a manner sufficient to perform at least the operations described herein. In some arrangements, the one or more processors may be shared by multiple servers (e.g., server A and server B may comprise or otherwise share the same processor, which, in some example arrangements, may execute instructions stored, or otherwise accessed, via different areas of memory). Alternatively or additionally, the one or more processors may be structured to perform or otherwise execute certain operations independent of one or more co-processors. In other example arrangements, two or more processors may be coupled via a bus to enable independent, parallel, pipelined, or multi-threaded instruction execution. Each processor may be implemented as one or more general-purpose processors, application specific integrated circuits (ASICs), field programmable gate arrays (FPGAs), digital signal processors (DSPs), or other suitable electronic data processing components structured to execute instructions provided by memory. The one or more processors may take the form of a single core processor, multi-core processor (e.g., a dual core processor, triple core processor, quad core processor), microprocessor, etc. In some arrangements, the one or more processors may be external to the apparatus, for example the one or more processors may be a remote processor (e.g., a cloud based processor). Alternatively or additionally, the one or more processors may be internal and/or local to the apparatus. In this regard, a given server or components thereof may be disposed locally (e.g., as part of a local server, a local computing system) or remotely (e.g., as part of a remote server such as a cloud based server). To that end, a “server” as described herein may include components that are distributed across one or more locations.

An exemplary system for implementing the overall system or portions of the arrangements might include a general purpose computing computers in the form of computers, including a processing unit, a system memory, and a system bus that couples various system components including the system memory to the processing unit. Each memory device may include non-transient volatile storage media, non-volatile storage media, non-transitory storage media (e.g., one or more volatile and/or non-volatile memories), etc. In some arrangements, the non-volatile media may take the form of ROM, flash memory (e.g., flash memory such as NAND, 3D NAND, NOR, 3D NOR), EEPROM, MRAM, magnetic storage, hard discs, optical discs, etc. In other arrangements, the volatile storage media may take the form of RAM, TRAM, ZRAM, etc. Combinations of the above are also included within the scope of machine-readable media. In this regard, machine-executable instructions comprise, for example, instructions and data which cause a general purpose computer, special purpose computer, or special purpose processing machines to perform a certain function or group of functions. Each respective memory device may be operable to maintain or otherwise store information relating to the operations performed by one or more associated servers, including processor instructions and related data (e.g., database components, object code components, script components), in accordance with the example arrangements described herein.

It should also be noted that the term “input devices,” as described herein, may include any type of input device including, but not limited to, a keyboard, a keypad, a mouse, joystick or other input devices performing a similar function. Comparatively, the term “output device,” as described herein, may include any type of output device including, but not limited to, a computer monitor, printer, facsimile machine, or other output devices performing a similar function.

It should be noted that although the diagrams herein may show a specific order and composition of method steps, it is understood that the order of these steps may differ from what is depicted. For example, two or more steps may be performed concurrently or with partial concurrence. Also, some method steps that are performed as discrete steps may be combined, steps being performed as a combined step may be separated into discrete steps, the sequence of certain processes may be reversed or otherwise varied, and the nature or number of discrete processes may be altered or varied. The order or sequence of any element or apparatus may be varied or substituted according to alternative arrangements. Accordingly, all such modifications are intended to be included within the scope of the present disclosure as defined in the appended claims. Such variations will depend on the machine-readable media and hardware systems chosen and on designer choice. It is understood that all such variations are within the scope of the disclosure. Likewise, software and web implementations of the present disclosure could be accomplished with standard programming techniques with rule based logic and other logic to accomplish the various database searching steps, correlation steps, comparison steps and decision steps.

It is also understood that any reference to an element herein using a designation such as “first,” “second,” and so forth does not generally limit the quantity or order of those elements. Rather, these designations can be used herein as a convenient means of distinguishing between two or more elements or instances of an element. Thus, a reference to first and second elements does not mean that only two elements can be employed, or that the first element must precede the second element in some manner.

The foregoing description of arrangements has been presented for purposes of illustration and description. It is not intended to be exhaustive or to limit the disclosure to the precise form disclosed, and modifications and variations are possible in light of the above teachings or may be acquired from this disclosure. The arrangements were chosen and described in order to explain the principals of the disclosure and its practical application to enable one skilled in the art to utilize the various arrangements and with various modifications as are suited to the particular use contemplated. Other substitutions, modifications, changes and omissions may be made in the design, operating conditions and arrangement of the arrangements without departing from the scope of the present disclosure as expressed in the appended claims.

What is claimed is:

1. A method comprising:
receiving, by one or more processors, a request for a risk score associated with a patient of one or more medical providers;
applying, by the one or more processors, scoring data associated with the patient to a risk predictive model to generate the risk score indicative of a probability of the patient to access in-person medical care at a medical provider within a temporal window that is subsequent to the one or more processors receiving the request for the risk score;
responsive to determining that the risk score satisfies a criteria, applying, by the one or more processors, at least a subset of the scoring data to an impactability predictive model to generate an impactability score indicative of a probability that the patient would not access the in-person medical care at the medical provider within the temporal window responsive to receiving a notification from the medical provider; and
sending, by the one or more processors, a message to a client device instructing the client device to present at least one of the risk score or the impactability score.

2. The method of claim 1, further comprising:
generating, by the one or more processors, a social determinant of health score by executing a second predictive model based on a first set of publicly available data, the social determinant of health score being indicative of a health status within a geographical region associated with the patient.

3. The method of claim 1, wherein the risk predictive model is trained with training data comprising at least one of medical data, medical image scores each indicative of a probability that a respective patient of a plurality of patients has a medical illness, social determinants of health scores each associated with a respective neighborhood, and clinician linkages each indicative of a degree of relationship between a plurality of physicians.

4. The method of claim 3, wherein the medical image scores are generated by a second predictive model based on a plurality of medical images and a plurality of medical diagnosis labels, each medical image of the plurality of medical images are associated with a respective medical diagnosis label of the plurality of medical diagnosis labels.

5. The method of claim 1, wherein the impactability predictive model is trained with training data comprising a plurality of identifiers associated with a plurality of patients, each identifier of the plurality of identifiers indicative of whether a respective patient of the plurality of patients accessed in-person medical care at one or more medical providers responsive to receiving the notification from the one or more medical providers.

6. The method of claim 1, wherein the scoring data comprises at least one of medical data associated with the patient, medical image scores associated with the patient, social determinants of health scores associated with the patient, or clinician linkages associated with the patient.

7. The method of claim 6, wherein clinical linkage indicates a degree of relationship between the one or more medical providers.

8. The method of claim 1, wherein the presentation comprises a graph depicting the impactability score on a first axis and a number of interventions on a second axis.

9. The method of claim 1, wherein the presentation comprises a graphical representation of an accuracy value associated with the risk predictive model or the impactability predictive model.

10. The method of claim 1, wherein the risk predictive model is trained with a first set of training data and the impactability predictive model is trained with a second set of training data different from the first set of training data.

11. A system comprising:
a server comprising a processor and a non-transitory computer-readable medium containing instructions that when executed by the processor causes the processor to perform operations comprising:
receive a request for a risk score associated with a patient of one or more medical providers;
apply scoring data associated with the patient to a risk predictive model to generate the risk score indicative of a probability of the patient to access in-person medical care at a medical provider within a temporal window that is subsequent to the processor receiving the request for the risk score;
responsive to determining that the risk score satisfies a criteria, apply at least a subset of the scoring data to an impactability predictive model to generate an impactability score indicative of a probability that the patient would not access the in-person medical care at the medical provider within the temporal window responsive to receiving a notification from the medical provider; and
send a message to a client device instructing the client device to present at least one of the risk score or the impactability score.

12. The system of claim 11, wherein the instructions further cause the processor to:
generate a social determinant of health score by executing a second predictive model based on a first set of publicly available data, the social determinant of health score being indicative of a health status within a geographical region associated with the patient.

13. The system of claim 11, wherein the risk predictive model is trained with training data comprising at least one of medical data, medical image scores each indicative of a probability that a respective patient of a plurality of patients has a medical illness, social determinants of health scores each associated with a respective neighborhood, and clinician linkages each indicative of a degree of relationship between a plurality of physicians.

14. The system of claim 13, wherein the medical image scores are generated by a second predictive model based on a plurality of medical images and a plurality of medical diagnosis labels, each medical image of the plurality of medical images are associated with a respective medical diagnosis label of the plurality of medical diagnosis labels.

15. The system of claim 11, wherein the impactability predictive model is trained with training data comprising a plurality of identifiers associated with a plurality of patients, each identifier of the plurality of identifiers indicative of whether a respective patient of the plurality of patients accessed in-person medical care at one or more medical providers responsive to receiving the notification from the one or more medical providers.

16. The system of claim 11, wherein the scoring data comprises at least one of medical data associated with the patient, medical image scores associated with the patient, social determinants of health scores associated with the patient, or clinician linkages associated with the patient.

17. The system of claim 16, wherein clinical linkage indicates a degree of relationship between the one or more medical providers.

18. The system of claim 11, wherein the presentation comprises a graph depicting the impactability score on a first axis and a number of interventions on a second axis.

19. The system of claim 11, wherein the presentation comprises a graphical representation of a accuracy value associated with the risk predictive model or the impactability predictive model.

20. The system of claim 11, wherein the risk predictive model is trained with a first set of training data and the impactability predictive model is trained with a second set of training data different from the first set of training data.
